(19) United States
(12) Patent Application Publication
(10) Pub. No.: US 2007/0238854 A1
(43) Pub. Date: Oct. 11, 2007

(54) Heterocyclic Fused Isothiazole and Isoselenazole Monomers and Conducting Polymers

(75) Inventors: Steffen Zahn, Pennsburg, PA (US); Michael Edward Ford, Coopersburg, PA (US); Joanne Cunningham, Kutztown, PA (US)

Correspondence Address: AIR PRODUCTS AND CHEMICALS, INC.;PATENT DEPARTMENT, 7201 HAMILTON BOULEVARD, ALLENTOWN, PA, 181951501, US

(73) Assignee: AIR PRODUCTS AND CHEMICALS, INC., Allentown, PA (US)

(21) Appl. No.: 11/733,335

(22) Filed: Apr. 10, 2007

Related U.S. Application Data
(60) Provisional application No. 60/790,982, filed on Apr. 11, 2006.

(51) Int. Cl. C07D 498/02; C08G 75/32

(52) U.S. Cl. 528/377; 548/121; 548/207; 528/406

(57) ABSTRACT

A composition of matter having the following structure: 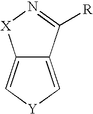
where X is S or Se; and Y is NH, O, S, or Se; and R is an H, a C₁₋₈F₃₋₁₇ primary, secondary, or tertiary fluoroalkyl, a C₁₋₂₀ primary, secondary or tertiary alkyl, or a halogen atom, and a composition of matter comprising polymerized units having the following structure: 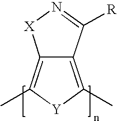
where X is S or Se; and Y is NH, O, S, or Se; and R is an H, a C₁₋₈F₃₋₁₇ primary, secondary, or tertiary fluoroalkyl, a C₁₋₂₀ primary, secondary or tertiary alkyl, or a halogen atom.

This Application claims the benefit of Provisional Application No. 60/790,982, filed on Apr. 11, 2006. The disclosure of this Provisional Application is hereby incorporated by reference.

### BACKGROUND OF THE INVENTION

Electrically conducting polymers have developed into a material of choice for a variety of organic optoelectronics applications. Such applications for optoelectronics include polymeric light emitting diodes (thin film displays), solid state lighting, organic photovolatics, advanced memory devices, organic field effect transistors, ultracapacitors, electroluminescent devices, printed electronics, conductors, lasers, and sensors.

One of the first of many electrically conducting polymers was polyacetylene and the discovery of conductivity in such polymer created substantial interest in other types of electrically conducting polymers. Recently, conjugated poly(thiophenes) and substituted thiophene derivatives have been discovered to have electrically conducting properties. A feature of these polymers is that they can be cast into films and doped with conventional p- and n-type dopants or the doped polymers can be cast into films and their electrical properties modified accordingly, thereby lending themselves suitable for use in a variety of optoelectronic applications.

Representative articles and patents illustrating thiophene monomers and electrically conducting polymers including thiophene and derivatives thereof are as follows:

U.S. Patent Application Publication No. US2004/00010115A1 discloses homopolymers and copolymers comprised of repeating units of thieno[3,4-b]thiophene for use in electroactive applications.

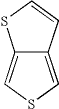

Copolymers can be formed with 3,4-ethylendioxythiophene, dithiophene, pyrrole, benzothiophene, and the like.

U.S. Pat. No. 6,645,401 discloses conjugated polymers of dithienothiophene (DTT) with vinylene or acetylene connecting groups as suitable for producing semiconductors or charge transport materials useful in electrooptical and electronic devices including field effect transistors, photovoltaic, and sensor devices. U.S. Pat. No. 6,585,914 discloses fluorocarbon-functionalized and/or heterocyclic modified poly(thiophenes) such as α,ω-diperfluorohexylsexithiophene for use in forming films which behave as n-type semiconductor. These poly(thiophenes) also can be used to form thin film transistors with FET mobility.

U.S. Pat. No. 6,676,857 discloses polymers having polymerized units of 3-substituted-4-fluorothiophene as liquid crystal materials for use in semiconductors, charge transport materials, electrooptical field effect transistors, photovoltaic and sensor devices.

U.S. Pat. No. 6,695,978 discloses polymers of benzo[b]thiophene and bisbenzo[b]-thiophene and their use as semiconductors and as charge transport materials in electrooptical devices.

U.S. Pat. No. 6,709,808 discloses image forming materials incorporating electrically conductive polymers based upon pyrrole-containing thiophene polymers and aniline containing polymers.

The article, Synthesis and Electronic Properties of Poly(2-phenyl-thieno[3,4-b]thiophene): A new Low Band Gap Polymer, Chem. Mater. 1999, 11, 1957-1958 discloses various thienothiophene polymers including poly(2-phenyl-thieno[3,4-b]thiophene) and poly(2-decyl-thieno[3,4-b]-thiophene) as conducting polymers.

The article, Poly(2-decyl-thieno[3,4-b]thiophene): a New Soluble Low-Band Gap Conducting Polymer, Synthetic Materials 1997, 84, 243-244 discloses various polymeric thienothiophenes including poly(2-decyl-thieno[3,4-b]thiophene and a process for preparing the polymer.

The article, Thienoisothiazoles, thienoisoselenazoles et selenoloisoselenazoles, Bull. Soc. Chim. Belg. 1980, 89, 773-778 discloses the sythesis of the monomer thienoisoselenazoles

The disclosure of the previously identified patents and patent applications are hereby incorporated by reference.

### BRIEF SUMMARY OF THE INVENTION

The instant invention relates to a composition of matter comprising: 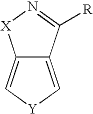
where X comprises S or Se; and Y comprises at least one of NH, O, S, or Se; and R comprises at least one of H, a C₁₋₈F₃₋₁₇ primary, secondary, or tertiary fluoroalkyl, a C₁₋₂₀ primary, secondary or tertiary alkyl, or a halogen atom.

In one aspect, the instant invention relates to a composition of matter comprising polymerized units comprising the following structure: 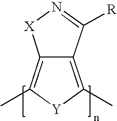
where X comprises S or Se; and Y comprises at least one of NH, O, S, or Se; and R comprises at least one of H, a C₁₋₈F₃₋₁₇ primary, secondary, or tertiary fluoroalkyl, a C₁₋₂₀ primary, secondary or tertiary alkyl, or a halogen atom.

The invention comprises heterocyclic fused isothiazole and isoselenazole based monomers and polymers comprising at least one of hydrogen, a C₁₋₈F₃₋₁₇ primary, secondary, or tertiary fluoroalkyl, or a C₁₋₂₀ primary, secondary or tertiary alkyl, or a halogen atom. Such monomers and polymers can be employed in electronic devides such as hole injection materials, charge transport materials, semiconductors, and/or conductors, in optical, electrooptical devices, polymeric light emitting diodes (PLED), electroluminescent devices, organic field effect transistors (FET or OFET), flat panel display applications (i.e. LCD's), radio frequency identification (RFID) tags, printed electronics, ultracapacitors, organic photovoltaics (OPV's), sensors, lasers, small molecule or polymer based memory devices, electrolytic capacitors, as hydrogen storage materials, among other applications.

Another aspect of this invention relates to dispersions containing at least one of the inventive monomers and polymers. If desired, the monomers and/or polymers can be dispersed within a medium comprising water and/or at least one solvent. These dispersions can be used for making conductive films as well as a precursor for another material.

Various advantages may be achieved by using the inventive monomers and polymers based upon heterocyclic fused isothiazoles and isoselenazoles and derivatives thereof of this invention. The advantages may include, without limitation:
an ability to produce conducting polymers having low workfunctions making them suited as a hole injecting material;
an ability to produce conducting polymers having a very low band gap making them suited as transparent conductors;
an ability to produce conducting polymers exhibiting electrical conduction without being p- or n- doped;
an ability to produce conducting polymers having a wide range of electronic applications;
an ability to produce a hole injection material having an excellent match of workfunction levels between the HIL material and the light emitting layer in an electroluminescent device;
an ability to produce both the oxidized and reduced forms of the polymers which results in the formation of a highly delocalized ionic polymer having high conductivity;
an ability to produce solution processable materials; and/or
an ability to produce environmentally stable semiconducting polymer.

### DETAILED DESCRIPTION OF THE INVENTION

The instant invention comprises a heterocyclic fused isothiazole and isoselenazole monomer having the following structure: 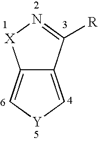
and conducting polymers formed by the polymerization of such monomers (polymerized units) where X comprises S or Se, and Y comprises at least one of NH, O, S, or Se. The numbers around the structure above indicate the 1-6 positions in the monomer. The R group in the 3-position as shown above. The “fused” isothiazole or isoselenazole means that a isothiazole or an isoselenazole share a common bond within the ring with a pyrrole, furan, thiophene or a selenophene, thereby fusing both ring-structures together. The invention also relates to polymers (e.g., conducting polymers) formed by the polymerization of such monomers (polymerized units). Examples of such monomers comprise at least one: 5H-Pyrrolo[3,4-d]isothiazole, Furo[3,4-d]isothiazole, Thieno[3,4-d]isothiazole, Selenolo[3,4-d]isothiazole, 5H-Pyrrolo[3,4-d]isoselenazole, Furo[3,4-d]isoselenazole, Thieno[3,4-d]isoselenazole, Selenolo[3,4-d]isoselenazole, and the oligomers and polymers comprised of polymerized units of 5H-Pyrrolo[3,4-d]isothiazole, Furo[3,4-d]isothiazole, Thieno[3,4-d]isothiazole, Selenolo[3,4-d]isothiazole, 5H-Pyrrolo[3,4-d]isoselenazole, Furo[3,4-d]isoselenazole, Thieno[3,4-d]isoselenazole, Selenolo[3,4-d]isoselenazole, and mixtures thereof. The structures for the monomers for these embodiments can comprise at least one of the following:

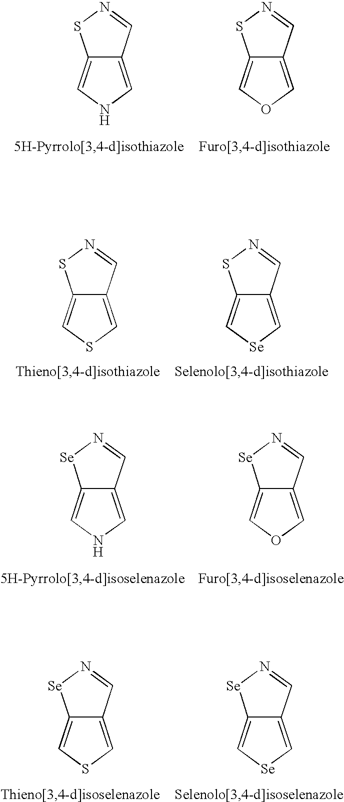

The instant invention also relates to derivatives of the heterocyclic fused isothiazole and isoselenazole that can be formed prior to or after the formation of the fully aromatic fused heterocyclic monomer. In one embodiment, such compositions of matter are represented by the compounds having the Formula A: 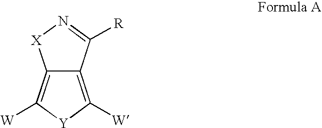
where X comprises S or Se; Y comprises at least one of NH, O, S, or Se; R is H, a C₁₋₈F₃₋₁₇ primary, secondary, or tertiary fluoroalkyl, a C₁₋₂₀ primary, secondary or tertiary alkyl, or a halogen atom; W and W′ can be any group or atom. In some embodiments W and W′ are independently selected from at least one member from the group of H, halogen atoms, MgCl, MgBr, MgI, ZnCl, ZnBr, ZnI, Sn(R′)₃ (where R′ is C₁₋₆ alkyl or —OC₁₋₆ alkyl), boronic acid, boronic ester, —CH═CHR″ (where R″ is H or C₁₋₆ alkyl), —OC₁₋₆ alkyl, —COOC₁₋₆ alkyl, —S—COR′″, —COR′″ (where R′″ is H or C₁₋₆ alkyl), —C≡CH, and polymerizable aromatic groups. Examples of C₁₋₈F₃₋₁₇ primary, secondary, or tertiary comprise at least one member from the group of trifluoromethyl, perfluoroethyl, perfluorobutyl, perfluoropropyl, 2,2,2-trifluoro-1-trifluoromethyl-ethyl and 2,2,2-trifluoro-1,1-bis-trifluoromethyl-ethyl, mixtures thereof, among others. Examples of C₁₋₂₀ primary, secondary or tertiary alkyls comprise at least one member from the group of isopropyl, tert-butyl, isopentyl, and 2-ethylhexyl, mixtures thereof, among others. Examples of halogen atoms comprise at least one of F, Cl, Br, I and the like. Examples of polymerizable aromatic groups comprise at least one member from the group of phenyl, naphthalene, pyrrole, dithiophene, thienothiophene, thiophene, mixtures thereof, among others.

The nature of the polymerization and the desired polymer can be controlled depending upon what constituents are selected for W and W′. Carbon-carbon bond forming polymerizations may be completed following procedures known to a person of ordinary skill in this art. Such procedures may include methods from the following incomplete list of well-known name reactions: Suzuki, Yamamoto, Heck, Stille, Sonogashira—Hagihara, Kumada-Corriu, Riecke, and McCullogh.

In one embodiment, monomers for producing homopolymers and copolymers are those where W and W′ are both H. In another embodiment, W, W′ and R are H.

In another aspect of the invention, the invention relates to electrically conducting oligomers and polymers comprised of polymerized units of monomers of Formula A. In one embodiment such a polymer may be represented by the Formula B: 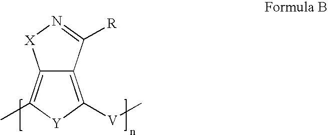
where X comprises S or Se; Y comprises NH, O, S, or Se; n is an integer; V comprise at least one of —CZ¹═CZ²— or —C≡C—; and Z¹ and Z² are independently H, F, Cl or CN. In one embodiment, n is from 2 to 50,000. Oligomers often have from about 2 to 10 units, that is, n is 2 to 10, and these products can be employed in the production of memory and field effect transistor devices. Polymers having from 11 to 50,000 units (e.g., n=11 to 50,000), or n is from 20 to 10,000 can be useful in preparing films such as hole injection materials in various electrooptical applications. (Unless otherwise specified, the use of the term polymer includes oligomers.)

In another aspect of the invention, the polymers may be represented by polymerized units of the Formula C: 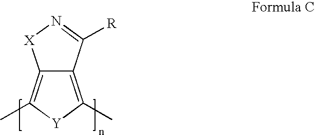
where X comprises S or Se; Y comprises at least one of NH, O, S, or Se; n is an integer as represented above and R comprises at least one of H, a C₁₋₂₀ primary, secondary or tertiary alkyl, a C₁₋₈F₃₋₁₇ primary, secondary or tertiary fluoroalkyl, or a halogen atom, wherein n is as described above. Examples of suitable monomers comprise at least one member from the group of thieno[3,4-d]isothiazole, selenolo[3,4-d]isothiazole, thieno[3,4-d]isoselenazole, and selenolo[3,4-d]isoselenazole, and polymers or oligomers comprising polymerized units of those monomers. This invention includes irregular polymers and regioregular polymers. Some embodiments that can provide low band gap are homopolymers of Formula B or C, and even lower band gap can be provided when the polymer displays high degrees of regioregularity, meaning that from about 50 to about 100 percent of the polymerizable units are bonded in a head to tail fashion as described by Richard D. McCullough in Handbook of Oligo- and Polythiophenes, (Ed. Denis Fichou), Wiley-VCH, Weinheim 1999, pages 1-41; hereby incorporated by reference. For example, for some polymers of the invention, a regioregular polymer is formed when the 6-position of a first polymerizable unit is bonded to the 4-position of a second polymerizable unit, and the 6-position of the second polymerizable unit is bonded to the 4-position of the third polymerizable unit, and so on. In other polymers of the invention, other uniform bonding patterns may form highly regioregular polymers of this invention. In other embodiments, about 70 percent or more of the polymerizable units are regioregular.

Monomers and polymers in which X comprises S and and Y comprises Se or S can provide relatively low band gaps. A more detailed discussion of band gaps and usage of low band gap materials has been described by Martin Pommerantz in Handbook of Conducting Polymers, (Ed. T. A. Skotheim, R. L. Elsenbaumer, and J. R. Reynolds), Marcel Dekker, New York 1998, pages 277-309; hereby incorporated by reference.

Synthesis of Thieno[3,4-d]isothiazole, for example, can be effected by a 3 step process as follows:

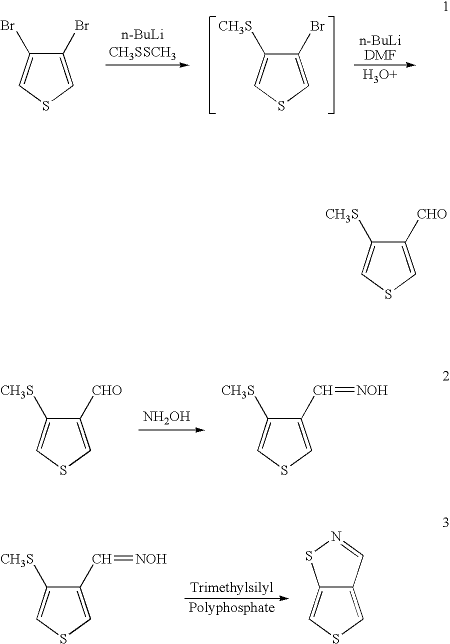

To prepare a isoselenazole-based thiophene derivative, dimethyl diselenide is used instead of the dimethyl disulfide shown above in step one. Additionally, the chemistries are somewhat similar for furan and selenophene (compared to thiophene), but these processes can be adapted for pyrrole.

Many of the derivatives of the respective monomers where W and W′ are other than H are formed post-formation of the monomers. In one such post-reaction, one or both hydrogen atoms may be replaced with other functional groups. Alternatively, some of the derivatives may be formed, ab initio, by converting thiophene to the derivative and then undergoing the foregoing three step reaction procedure wherein the W and W′ are compatible with the chemistries outlined in steps one through three above.

Polymerization of thieno[3,4-d]isothiazole monomer can be effected utilizing an aqueous phase polymerization method wherein the monomer thieno[3,4-d]isothiazole, a polyanion and an oxidant are reacted in the presence of water under reaction conditions sufficient to form the homopolymer, e.g., poly(thieno[3,4-d]isothiazole). By this polymerization process, the resulting monomer may be polymerized and doped in a single step.

The amount of polyanion and oxidant to be employed in the aqueous polymerization method may vary and can be determined for any given polymerization without undue experimentation. For example the weight ratio of thieno[3,4-d]isothiazole monomer to a desired polyanion typically ranges from about 0.001 to about 50, usually about 0.05 to about 2.0. The weight ratio of thieno[3,4-d]isothiazole monomer to a suitable oxidant typically ranges from about 0.01 to about 10, usually 0.1 to 2.0. For example, when ferric sulfate is used as the oxidant the amount used ranges from about 0.1 to about 5 of thieno[3,4-d]isothiazole. The nature of the oxidant may be varied in order to address variants in the ionization potential of the utilized monomers. Various Fe(II)/Fe(III) couplets can display different potentials depending on their respective ligands, e.g., FeCl₃; Fe₂(S₂O₈)₃; and Fe(1,10-phenanthroline)₃. If weaker oxidants are employed Cu based couplets may be used. If stronger oxidants are useful Co based couplets may be used.

Strong oxidants can be employed in the polymerization process. Persulfates and iron (III) salts of organic acids and inorganic acids containing organic residues are useful because they generally are not corrosive. Examples of suitable iron (III) salts of organic acids comprise at least one member from the group of Fe(III) salts of C₁₋₃₀ alkyl sulfonic acids, such as, methane or dodecane sulfonic acid; aliphatic C₁₋₂₀ carboxylic acids, such as 2-ethylhexylcarboxylic acid, aliphatic perfluorocarboxylic acids, such as trifluoroacetic acid and perfluorooctanoic acid; aliphatic dicarboxylic acids, such as, oxalic acid, and aromatic, optionally C₁₋₂₀-alkyl-substituted sulfonic acids, such as, benzenesulfonic acid, p-toluene-sulfonic acid, and dodecyl benzenesulfonic acid. Specific examples of iron salts include FeCl₃, Fe₂(SO₄)₃, Fe(ClO₄)₃ and Fe₂(S₂O₈)₃, Other oxidants include H₂O₂, K₂Cr₂O₇, ammonium persulfate, potassium permanganate, copper tetrafluoroborate, iodine, ozone, air and oxygen.

Suitable polyanions comprise at least one anion of a polycarboxylic acid, such as polyacrylic acid, polymethacrylic acid, perfluoronated ion-exchange polymers, perfluorosulfonate ionomers, e.g. (Nafion® products available from the DuPont Company), functionalized polyarylethers, polymaleic acid, and polymeric sulfonic acids, such as polystyrene sulfonic acid, polyvinyl sulfonic acid and polystryrene sulfonic acid-acrylonitrile copolymers. The polycarboxylic and polysulfonic acids may also be copolymers of vinyl carboxylic and vinyl sulfonic acids with other monomers, such as, acrylates and styrene. The molecular weight of the acids supplying the polyanions may be in the range from about 1,000 to about 500,000, or from about 2000 to about 500,000, or about 200,000.

Monomers of the Formula A can be employed in metal-catalyzed polymerizations. For examples see, Heck, Chem. Rev. 2000, 100, 3009-3066; Stille, Chem. Rev. 2003, 103, 169-196; Suzuki, Chem. Rev. 1995, 95, 2457-2483; Sonogashira—Hagihara, Chem. Rev. 2003, 103, 1979-2017; and Kumada-Corriu, Chem. Rev. 2002, 102, 1359-1469; the foregoing are hereby incorporated herein as references. Conditions can vary depending on the nature the W and W′ substituents. Polymers having lower band gaps can be achieved in Riecke-type and McCullogh-type polymerizations, which provide polymers having high degrees of regioregularity. These polymerizations are described in U.S. Pat. Nos. 5,756,653; 6,602,974; 6,166,172; US Pub. No. 2004/0024171 A1; and 2004/0030091 A1; and EP 1 028 136 A2; Richard D. McCullough in Handbook of Oligo- and Polythiophenes, (Ed. Denis Fichou), Wiley-VCH, Weinheim 1999, pages 1-41, all of which are incorporated herein by reference.

A method for preparing oligomers and polymers such as poly(thieno[3,4-d]isothiazole), comprises an electrochemical process wherein thieno[3,4-d]isothiazole is polymerized in an electrochemical cell using a three electrode configuration. A suitable three electrode configuration comprises a button working electrode selected from the group consisting of platinum, gold and vitreous carbon button working electrodes, a platinum flag counter electrode and an Ag/Ag+ non-aqueous reference electrode. Suitable electrolytes comprise at least one member selected from the group consisting of tetrabutylammonium perchlorate/acetonitrile, lithium triflate/acetonitrile and tetrabutylammonium hexafluorophosphate/acetonitrile.

Films of oligomers or polymers of this invention in which the R comprises at least one of H, C₁₋₂₀ primary, secondary or tertiary alkyl, C₁₋₈F₃₋₁₇ primary, secondary or tertiary fluoroalkyls, and halogen atoms, for example, substituted thieno[3,4-d]isothiazole oligomers and polymers, may be doped with conventional p- and n- type dopants post polymerization of the respective monomers. The doping process typically comprises treating the film semiconductor material with an oxidizing or a reducing agent in a redox reaction to form delocalized ionic centers in the material, with the corresponding counterions derived from the applied dopants. Doping methods comprise, for example, exposure to a doping vapor at atmospheric or at a reduced pressure, electrochemical doping in a solution containing a dopant, bringing the dopant in contact with the semiconductor material to be thermally diffused, and ion-implantantion of the dopant into the semiconductor material.

Conductive polymeric films having holes (p-doped) can be formed via conventional p-dopants which comprise at least one of halogen atoms, e.g., I₂, Cl₂, Br₂, ICl, ICl₃, IBr and IF, Lewis acids, e.g., PF₅, AsF₅, SbF₅, BF₃, BCl₃, SbCl₅, BBr₃ and SO₃, protonic acids, organic acids, or amino acids, e.g., HF, HCl, HNO₃, H₂SO₄, HClO₄, FSO₃H and ClSO₃H, transition metal compounds, e.g., FeCl₃, Fe(OCl)₃, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnX₃ wherein Ln is a lanthanoid and X is an anion, e.g., Cl⁻, Br⁻, I⁻, I₃⁻, HSO₄⁻, SO₄²⁻, NO₃⁻, ClO₄⁻, BF₄⁻, B₁₂F₁₂²⁻, PF₆⁻, AsF₆⁻, SbF₆⁻, FeCl₄⁻, Fe(CN)₆³⁻, and anions of various sulfonic acids, such as aryl-SO₃⁻. Also, O₂, as well as O₃, may be used.

Conductive polymeric films employing electrons as carriers as in n-doped polymeric films utilize conventional n-dopants which can comprise the alkali metals (e.g., Li, Na, K, Rb, and Cs), and alkaline-earth metals e.g., Ca, Sr, and Ba.

When R comprises at least one of H, C₁₋₂₀ primary, secondary or tertiary alkyl, C₁₋₁₈F₃₋₁₇ primary, secondary or tertiary fluoroalkyl, or a halogen atom substituted thieno[3,4-d]isothiazole, such as 3-(methyl)-thieno[3,4-d]isothiazole and its derivatives, those monomers can be copolymerized with other polymerizable monomers capable of forming electrically conductive polymers. Such monomers comprise at least one of benzo- and bisbenzothiophenes, thiophene, dithienothiophene, pyridylthiophenes, substituted thiophenes, substituted thieno[3,4-b]thiophenes, dithieno[3,4-b:3′,4′-d]thiophene, pyrrole, bithiophene, substituted pyrroles, phenylene, substituted phenylenes, naphthalene, substituted naphthalenes, biphenyl and terphenyl and their substituted derivatives, phenylene vinylene and substituted phenylene vinylene. Other polymerizable monomers that may be copolymerized with monomers of this invention are described in U.S. Pat. No. 4,959,430, and U.S. Pat. No. 4,910,645; hereby incorporated by reference.

When processing oligomers and polymers of thieno[3,4-d]isothiazole and oligomers and polymers of substituted thieno[3,4-d]isothiazole such as 2-(methyl)-thieno[3,4-d]isothiazole and derivatives, additives such as at least one of ethylene glycol, diethylene glycol, mannitol, propylene 1,3-glycol, butane 1,4-glycol, N-methylpyrrolidone, sorbitol, glycerol, propylene carbonate and other appropriate high boiling organics, can be added to dispersions in order to modify the conductivity of the films prepared from these dispersions. Other common additives for tailoring electrically conductive polymers can be employed as desired and these comprise at least one of antioxidants, UV stabilizers, surfactants, and conductive fillers such as particulate copper, silver, gold, indium tin oxide, carbon nanotubes, nickel, aluminum, carbon black and the like. Non-conductive fillers such as talc, mica, wollastonite, silica, clay, TiO₂, dyes, pigments and the like can also be incorporated to promote specific properties such as increased modulus, surface hardness, surface color and the like.

Another aspect of the invention relates to dispersions containing the foregoing additives, components, compounds and at least one of the inventive monomers, oligomers and polymers. If desired, the monomers, oligomers, and/or polymers can be dispersed within a medium comprising water and/or at least one solvent. These dispersions can further comprise at least one acid source such as at least one member selected from the group consisting of carboxylic acids, phosphonic acid, phosphoric acids and sulfonic acids. The pKa of these dispersions is typically less than about 4.0. While any suitable amounts can be used, the monomer, oligomer and/or polymer can comprise about 0 to about 99wt. % of the dispersion; water and/or solvent can comprise greater than 0 to about 6wt. % of the dispersion, and the acid source can comprise greater than 0 to about 6wt. % of the dispersion. These dispersions can be used for making conductive films as well as a precursor for another material.

The following examples are provided to illustrate various embodiments and comparisons and are not intended to restrict the scope of the invention.

### EXAMPLE 1

The compound thieno[3,4-d]isothiazole can be prepared by a three step procedure in the manner described. Step 1 was used to prepare 4-methylthio-3-thiophenecarboxaldehyde, Step 2 was used to prepare 4-Methylthio-3-thiophenecarboxaldehyde oxime, and Step 3 is used to prepare thieno[3,4-d]isothiazole.

### Step 1

### Preparation of 4-methylthio-3-thiophenecarboxaldehyde

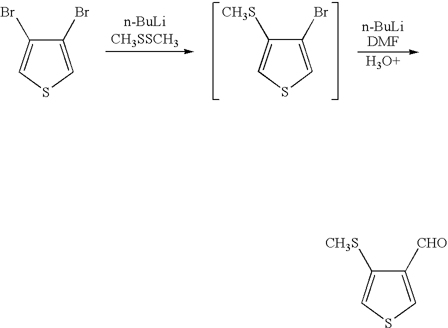

In a typical procedure, 40 mL of anhydrous THF in a 100 mL flask was cooled to −78C under nitrogen with stirring. After 2 hr. at −78C, 2.1 mL of 2.6 M n-BuLi in hexanes (0.00525 mole) was added via syringe. Simultaneously, 1.212 gm (0.00501 mole) of 3,4-dibromothiophene was weighed into 15 mL flask and then purged with nitrogen for 2 hr. THF, 12 mL, was then added to the 15 mL flask via syringe. After the n-BuLi/hexanes/THF solution had been at −78C for 20 min., the solution of 3,4-dibromothiophene was added dropwise via cannula; this addition took 3 minutes. The resulting solution was stirred at −78C for 5 min., after which time 0.47 gm (0.005 mole) of dimethyl disulfide was added dropwise via syringe. The latter addition required ca 3 min.

Meanwhile, a second 100 mL flask was purged with nitrogen, and 35 mL THF added. After cooling to −78C with stirring, it was maintained at −78C for 2.25 hr prior to addition of 2.1 mL of 2.6 M n-BuLi in hexanes. The solution of 4-methylthio-3-bromothiophene prepared above was removed from its cooling bath at the same time as the n-BuLi was added to the second 100 mL flask. After the second n-BuLi/hexanes/THF solution had stirred at −78C for 20 min., the solution of 4-methylthio-3-bromothiophene was transferred to it via cannula. This took 15 min. After an additional 15 min. at −78C, 0.45 mL DMF (0.0058 mole; 1.16 eq) was added via syringe. The reaction was maintained at −78C for an additional 2 hr., after which it was allowed to warm to ambient. It was quenched with 30 mL of 1 M HCl and partitioned between toluene and saturated sodium chloride solution. After drying (anhydrous MgSO₄), evaporation of the toluene provided crude 4-methylthio-3-thiophenecarboxaldehyde.

### Step 2

### Preparation of 4-Methylthio-3-thiophenecarboxaldehyde Oxime

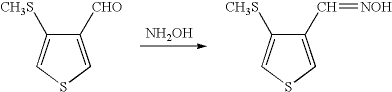

In a typical procedure, 0.484 gm (0.00306 mole) of 4-methylthio-3-thiophene-carboxaldehyde was weighed into 100 mL flask. A solution of 0.495 gm (0.00619 mole; 2.0 equiv.) 50% aqueous sodium hydroxide in 40 mL absolute ethanol was added. Subsequently, hydroxylamine hydrochloride (0.423 gm; 0.00609 mole; 2.0 equiv.) was added along with 7 mL of deionized water. The resulting suspension was heated to 50C with stirring; all solids were dissolved by the time 50C was attained. After heating at 50C for 2 hr, the reaction was cooled to ambient, partitioned between ethyl acetate and saturated aqueous sodium chloride. Drying (MgSO₄) and evaporation yielded crude 4-methylthio-3-thiophenecarboxaldehyde oxime.

### Step 3

### Preparation of Thieno[3,4-d]isothiazole

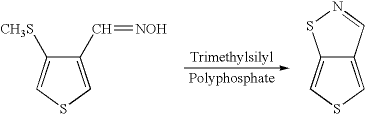

In a typical procedure, 0.147 gm (0.00085 mole) of 4-thiomethyl-3-thiophene-carboxaldehyde oxime is weighed into a 50 mL flask, to which is then added 11.13 gm of trimethylsilyl polyphosphate. Toluene, 20 mL, is also added, and the resulting mixture is stirred at room temperature under nitrogen. A homogeneous solution is formed after ca 15-30 min. The resulting solution is stirred at ambient under nitrogen for 70 hr. At the end of this time, the mixture is poured onto ice water and the pH adjusted to 7-8 with 10% aqueous sodium hydroxide solution. The resulting mixture is partitioned between ethyl acetate and saturated aqueous sodium chloride. After drying (anhydrous MgSO₄), evaporation provided crude thieno[3,4-d]isothiazole.

### EXAMPLE 2

### Electrochemical Polymerization of thieno[3,4-d]isothiazole

Thieno[3,4-d]isothiazole is dissolved in 100 mM tetrabutylammonium hexafluorophosphate/acetonitrile solution to a concentration of 10 mM monomer and is electrochemically polymerized employing a 3-electrode configuration, using a platinum button working electrode (2 mm diameter), platinum flag counter electrode (1 cm²), and a Ag/Ag+ nonaqueous reference electrode. The applied potential is cycled between 1.6V and −0.8V at a rate of 100 mV/sec.

Polymerization is apparent from the current response increase in regular intervals at a lower redox potential upon repetitive scans.

### EXAMPLE 3

### Aqueous Synthesis of Poly(Thieno[3,4-d]isoselenazole)

50 mg of Thieno[3,4-d]isoselenazole and 830 mg of 18% poly(styrenesulfonic acid) water solution in 10 ml of deionized water is added to a 25 ml 1-neck flask. The mixture is stirred at 600 rpm. 113.0 mg (0.48 mmol) of (NH₄)₂S₂O₈ and 2 mg of Fe₂(SO₄)₃ were added to the reaction flask. The oxidative polymerization is carried out in excess of one hour. After polymerization, the aqueous solution is purified by ion exchange columns (Amberlite® IR-120 and MP62) resulting in a deep black aqueous poly(Thieno[3,4-d]isoselenazole)/poly(styrene sulfonic acid) dispersion. Transparent films are prepared by spin coating the poly(Thieno[3,4-d]isoselenazole)/poly(styrene sulfonic acid) mixture onto glass substrates at 1,000 rpm yielding an electrically conductive surface.

### EXAMPLE 4

### Aqueous Synthesis of Poly(Thieno[3,4-d]isoselenazole)

50 mg of Thieno[3,4-d]isoselenazole and 5.55 g of 18% poly(styrenesulfonic acid) water solution in 45 ml of deionized water is added to a 100 ml 1 -neck flask. The mixture is stirred at 1200 rpm. 300 mg (1.98 mmol) of Fe₂(SO₄)₃ dissolved in 7 mL deionized water are added to the reaction flask. The oxidative polymerization is carried out in excess of one hour. After polymerization, the aqueous solution is purified by ion exchange columns, resulting in a deep black aqueous Poly(Thieno[3,4-d]isoselenazole)/poly(styrene sulfonic acid) dispersion. Transparent films are prepared by spin coating the poly(Thieno[3,4-d]isoselenazole)/poly(styrene sulfonic acid) mixture onto glass substrates at 1,000 rpm yielding an electrically conductive surface.

### EXAMPLE 5

### Aqueous Synthesis of Poly(Thieno[3,4-d]isoselenazole)

50 mg of Thieno[3,4-d]isoselenazole and 8.4 g of 12% NAFION® perfluorinated ion-exchange resin water dispersion in 42 ml of deionized water is added to a 100 ml 1-neck flask (NAFION® is a federally registered trademark of E. I. DuPont deNemours and Company, Wilmington, Del.). The mixture is stirred at 1200 rpm. 300 mg (1.98 mmol) of Fe₂(SO₄)₃ dissolved in 7 mL deionized water are added to the reaction flask. The oxidative polymerization is carried out in excess of one hour. After polymerization, the aqueous solution is purified by ion exchange columns, resulting in a deep black aqueous poly(Thieno[3,4-d]isoselenazole)/Nafion® dispersion. Transparent films are prepared by spin coating the poly(Thieno[3,4-d]isoselenazole)/Nafion® mixture onto glass substrates at 1,000 rpm yielding an electrically conductive surface.

### EXAMPLE 6

### Aqueous Synthesis of Poly(Thieno[3,4-d]isoselenazole)

50 mg of Thieno[3,4-d]isoselenazole and 8.4 g of 12% NAFION® perfluorinated ion-exchange resin water dispersion in 42 ml of deionized water is added to a 100 ml 1-neck flask. The mixture is stirred at 1200 rpm. 113.0 mg (0.48 mmol) of (NH₄)₂S₂O₈ and 2 mg of Fe₂(SO₄)₃ are added to the reaction flask. The oxidative polymerization is carried out in excess of one hour. After polymerization, the aqueous solution is purified by ion exchange columns (Amberlite® IR-120 and MP62) resulting in a deep black aqueous poly(Thieno[3,4-d]isoselenazole)/Nafion® dispersion. Transparent films are prepared by spin coating the poly(Thieno[3,4-d]isoselenazole)/poly(styrene sulfonic acid) mixture onto glass substrates at 1,000 rpm yielding an electrically conductive surface.

### EXAMPLE 7

### Solvent (in-situ) Synthesis of Poly(Thieno[3,4-d]isoselenazole)

280 mg of Thieno[3,4-d]isoselenazole are dissolved in 15 mL anhydrous n-butanol. 2.25 g (3.3 mmol) of iron (III) p-toluenesulfonate hexahydrate dissolved in 5 mL of anhydrous n-butanol is added to the monomer solution resulting in a deep red solution. The mixture is drop cast on glass substrates and allowed to dry. The dried film is cured at temperatures up to 120° C. for up to 15 minutes. The resulting film is conductive as determined by the four-point-probe measurement.

While the invention has been described with reference to certain aspects, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, the invention shall not be limited to any aspect contemplated for carrying out this invention, but that the invention will include all subject matter falling within the scope of the appended claims.

1. A composition of matter comprising: 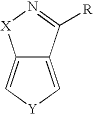
where X comprises S or Se; and Y comprises at least one member selected from the group consisting of NH, O, S and Se; and R comprises at least one member selected from the group consisting of H, a C₁₋₈F₃₋₁₇ primary, secondary, or tertiary fluoroalkyl, a C₁₋₂₀ primary, secondary or tertiary alkyl, and a halogen atom.

2. The composition of matter of claim 1, further comprising the structure: 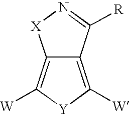
where W and W′ are independently selected from the group consisting of H, halogen atoms, MgCl, MgBr, MgI, ZnCl, ZnBr, ZnI, Sn(R′)₃, boronic acid, boronic ester, —CH═CHR″, —OC₁₋₆ alkyl, —COOC₁₋₆ alkyl, —S—COR′″, —COR′″—C≡CH, and polymerizable aromatic groups; wherein R′ comprises C₁₋₆ alkyl or —OC₁₋₆ alkyl; R″ is H or C₁₋₆ alkyl; and R′″ is H or C₁₋₆alkyl.

3. The composition of matter of claim 1 comprising at least one member selected from the group consisting of:

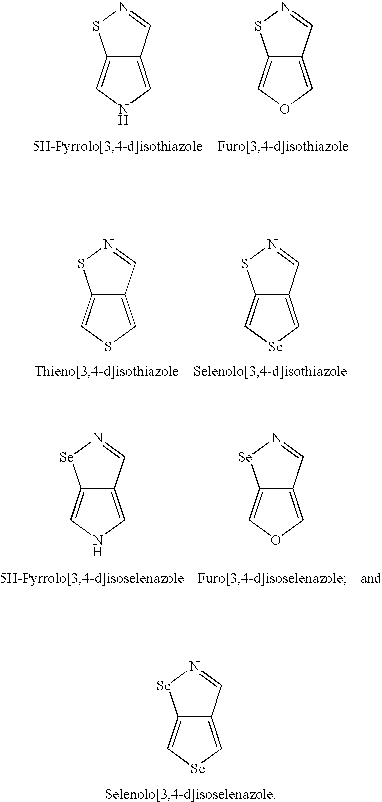

4. The composition of claim 1 wherein X comprises S and Y comprises S.

5. A composition of matter comprising polymerized units comprising the following structure: 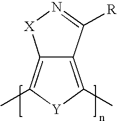
where X comprises S or Se; and Y comprises at least one member selected from the group consisting of NH, O, S, or Se; and R is an H, a C₁₋₈F₃₋₁₇ primary, secondary, or tertiary fluoroalkyl, a C₁₋₂₀ primary, secondary or tertiary alkyl, or a halogen atom.

6. A dispersion comprising the polymer of claim 5, at least one member selected from the group consisting of water and at least one solvent, and at least one acid in monomeric or polymeric form with pKa of less than about 4.

7. The dispersion of claim 5 wherein the acid comprises at least one member selected from the group consisting of carboxylic acids, phosphonic acid, phosphoric acids and sulfonic acids.
